(19) United States
(12) Patent Application Publication
(10) Pub. No.: US 2005/0176739 A1
(43) Pub. Date: Aug. 11, 2005

(54) Therapeutic agent for eating disorders

(76) Inventors: Takuji Hara, Ube-shi (JP); Yumiko Ishikawa, Mishima-shi (JP); Tetsuya Ryomoto, Ube-shi (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 11/104,623

(22) Filed: Apr. 13, 2005

Related U.S. Application Data
(63) Continuation of application No. 10/111,446, filed on Apr. 25, 2002.

(30) Foreign Application Priority Data
Oct. 29, 1999 (JP) 310138/99

(51) Int. Cl. A61K 31/522

(52) U.S. Cl. 514/263.24; 514/263.34

(57) ABSTRACT

The present invention provides a therapeutic agent for eating disorders, comprising, as an active ingredient, a xanthine derivative represented by formula (I): 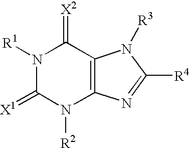
wherein R¹, R² and R³ independently represent hydrogen, lower alkyl, lower alkenyl or lower alkynyl; R⁴ represents cycloalkyl, —(CH₂)ₙ—R⁵ or the like; and X¹ and X² independently represent O or S, or a pharmaceutically acceptable salt thereof.

### TECHNICAL FIELD

The present invention relates to therapeutic agents for eating disorders.

### BACKGROUND ART

Most of the compounds represented by formula (I) shown below and compounds related thereto are known compounds, and their adenosine A₂-receptor antagonism anti-Parkinson's disease action, anti-depressive action, anti-asthmatic action, inhibitory action on bone absorption, action on central excitation and inhibitory action on neurodegeneration are known [JP 47-26516 B, J. Med. Chem., 34, 1431 (1991), J. Med. Chem., 36, 1333 (1993), WO 92/06976, JP 6-211856 A, JP 6-239862 A, WO 95/23165, JP 6-1655-9 A, WO 94/01114 and WO 99/12546].

However, it is not known that said compounds have aperitive activity.

### DISCLOSURE OF THE INVENTION

An object of the present invention is to provide excellent therapeutic agents for eating disorders.

The present invention relates to those described below.

(1) A therapeutic agent for eating disorders comprising, as an active ingredient, a xanthine derivative represented by formula (I): 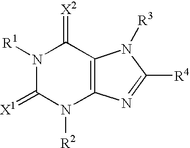
wherein R¹, R² and R³ independently represent hydrogen, lower alkyl, lower alkenyl or lower alkynyl; R⁴ represents cycloalkyl, —(CH₂)ₙ—R⁵ (wherein R⁵ represents substituted or unsubstituted aryl, or a substituted or unsubstituted heterocyclic group, and n is an integer of 0 to 4), or the following group: 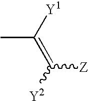
wherein Y¹ and Y² independently represent hydrogen, halogen or lower alkyl, and Z represents substituted or unsubstituted aryl, the following group: 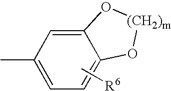
(wherein R⁶ represents hydrogen, hydroxy, lower alkyl, lower alkoxy, halogen, nitro or amino, and m is an integer of 1 to 3), or a substituted or unsubstituted heterocyclic group; and X¹ and X² independently represent O or S, or a pharmaceutically acceptable salt thereof.

(2) The therapeutic agent for eating disorders comprising, as an active ingredient, the xanthine derivative according to the above (1) wherein X¹ and X² are O, or a pharmaceutically acceptable salt thereof.

(3) The therapeutic agent for eating disorders comprising, as an active ingredient, the xanthine derivative according to the above (1) or (2) wherein R⁴ is the following group: 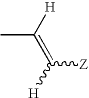
wherein Z has the same meaning as defined above, or a pharmaceutically acceptable salt thereof.

(4) An aperitive comprising, as an active ingredient, the xanthine derivative according to any one of the above (1) to (3) or a pharmaceutically acceptable salt thereof.

(5) Use of the xanthine derivative according to any one of the above (1) to (3) or a pharmaceutically acceptable salt thereof for the production of a therapeutic agent for eating disorders.

(6) Use of the xanthine derivative according to any one of the above (1) to (3) or a pharmaceutically acceptable salt thereof for the production of an aperitive.

(7) A method for therapeutically treating or preventing eating disorders, which comprises administering an effective amount of the xanthine derivative according to any one of the above (1) to (3) or a pharmaceutically acceptable salt thereof.

(8) A method for therapeutically treating or preventing anorexia, which comprises administering an effective amount of the xanthine derivative according to any one of the above (1) to (3) or a pharmaceutically acceptable salt thereof.

Hereinafter, the compound represented by formula (I) is referred to as compound (I).

In the definition of compound (I), the lower alkyl and the lower alkyl moiety in the lower alkoxy mean a straight-chain or branched C₁-C₆ alkyl group such as methyl, ethyl, propyl, isopropyl, butyl, isobutyl, sec-butyl, tert-butyl, pentyl, neopentyl or hexyl; the lower alkenyl means a straight-chain, or branched C₂-C₆ alkenyl group such as vinyl, allyl, methacryl, crotyl, 3-butenyl, 2-pentenyl, 4-pentenyl, 2-hexenyl or 5-hexenyl; the lower alkynyl means a straight-chain or branched C₂-C₆ alkynyl group such as ethynyl, propargyl, 2-butynyl, 3-butynyl, 2-pentynyl, 4-pentynyl, 2-hexynyl, 5-hexynyl or 4-methyl-2-pentynyl; the aryl means phenyl or naphthyl; the cycloalkyl means a C₃-C₈ cycloalkyl group such as cyclopropyl, cyclobutyl, cyclopentyl, cyclohexyl, cycloheptyl or cyclooctyl; examples of the heterocyclic group are furyl, thienyl, pyrrolyl, pyranyl, thiopyranyl, pyridyl, thiazolyl, imidazolyl, pyrimidinyl, triazinyl, indolyl, quinolyl, purinyl and benzothiazolyl; and the halogen includes fluorine, chlorine, bromine and iodine. The substituted aryl and the substituted heterocyclic group have 1, to 3 independently-selected substituents such as lower alkyl, hydroxy, substituted or unsubstituted lower alkoxy, halogen, nitro, amino, lower alkylamino, di(lower alkyl)amino, trifluoromethyl, trifluoromethoxy, aralkyl, aralkyloxy, aryl, aryloxy, lower alkanoyl, lower alkanoyloxy, aroyl, aroyloxy, arylalkanoyloxy, carboxy, lower alkoxycarbonyl, lower alkylcarbamoyl, di(lower alkyl)carbamoyl, sulfo, lower alkoxysulfonyl, lower alkylsulfamoyl or di(lower alkyl)sulfamoyl. The lower alkyl and the alkyl moiety in the lower alkoxy, lower alkylamino, di(lower alkyl) amino, lower alkanoyl, lower alkanoyloxy, lower alkoxycarbonyl, lower alkylcarbamoyl, di(lower alkyl)carbamoyl, lower alkoxysulfonyl, lower alkylsulfamoyl and di(lower alkyl)sulfamoyl have the same meaning as the lower alkyl defined above. The halogen has the same meaning as defined above. The aryl and the aryl moiety in the aryloxy have the same meaning as the aryl defined above. Examples of the aralkyl and the aralkyl moiety in the aralkyloxy are benzyl and phenethyl. Examples of the aroyl and the aroyl moiety in the aroyloxy are benzoyl and naphtoyl. Examples of the arylalkyl moiety in the arylalkanoyloxy are benzyl and phenethyl. Examples of the substituents for the substituted lower alkoxy are hydroxy, lower alkoxy, halogen, amino, azido, carboxy and lower alkoxycarbonyl. The alkyl moiety in the lower alkoxy and lower alkoxycarbonyl has the same meaning as the lower alkyl defined above, and the halogen has the same meaning as the halogen defined above.

The pharmaceutically acceptable salts of compound (I) include pharmaceutically acceptable acid addition salts, metal salts, ammonium salts, organic amine addition salts and amino acid addition salts.

The pharmaceutically acceptable acid addition salts of compound (I) include inorganic acid addition salts such as hydrochloride, sulfate and phosphate, and organic acid addition salts such as acetate, maleate, fumarate, tartrate, citrate and methanesulfonate; the pharmaceutically acceptable metal salts include alkali metal salts such as sodium salt and potassium salt, alkaline earth metal salts such as magnesium salt and calcium salt, aluminum salt, and zinc salt. The pharmaceutically acceptable ammonium salts include ammonium and tetramethylammonium. The pharmaceutically acceptable organic amine addition salts include a salt with morpholine or piperidine; and the pharmaceutically acceptable amino acid addition salts include a salt with lysine, glycine or phenylalanine.

Compound (I) including novel compounds can be produced by the methods disclosed in the above-mentioned publications or according to the methods. The desired compound in the process can be isolated and purified by purification methods conventionally used in synthetic organic chemistry, such as filtration, extraction, washing, drying, concentration, recrystallization or various kinds of chromatography.

In the case where a salt of compound (I) is desired and it is produced in the form of a desired salt, it may be subjected to purification as such. In the case where compound (I) is produced in the free form and its salt is desired, it is dissolved or suspended in a suitable solvent, and then an acid or a base may be added thereto to form the salt.

Compound (I) and pharmaceutically acceptable salts thereof may be in the form of an adduct with water or various solvents, which can also be used as the therapeutic agent of the present invention.

Some of compounds (I) have optical isomers, and all potential stereoisomers and mixtures thereof can also be used as the therapeutic agent of the present invention.

Examples of compound (I) are shown in Table 1.

TABLE 1
Compound No.

| | |
|---|---|
| 1 | 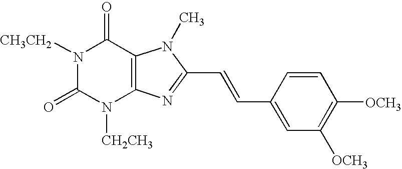 |
| 2 | 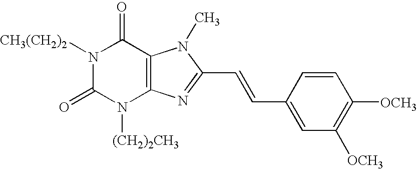 |
| 3 | 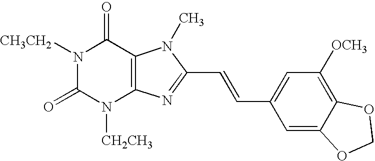 |
| 4 | 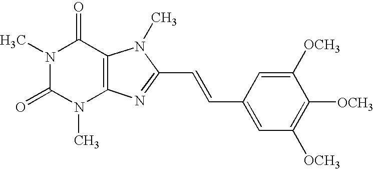 |

Compound 1: (E)-8-(3,4-dimethoxystyryl)-1,3-diethyl-7-methylxanthine (JP 6-211856 A)

Melting point: 190.4-191.3° C.

Elemental analysis: C₂₀H₂₄N₄O₄

Calcd. (%): C 62.48, H 6.29; N 14.57

Found (%): C, 62.52; H, 6.53; N, 14.56

IR(KBr) νmax(cm⁻¹): 1697, 1655, 1518

NMR(CDCl₃, 270 MHz) δ(ppm): 7.74(1H, d, J=15.5 Hz), 7.18(1H, dd, J=8.3, 1.9 Hz), 7.08(1H, d, J=1.9 Hz), 6.89(1H, d, J=8.3 Hz), 6.77(1H, d, J=15.5 Hz), 4.21(2H, q, J=6.9 Hz), 4.09(2H, q, J=6.9 Hz), 4.06(3H, s), 3.96(3H, s), 3.93(3H, s), 1.39(3H, t, J=6.9 Hz), 1.27(3H, t, J=6.9 Hz)

Compound 2: (E)-8-(3,4-dimethoxystyryl)-7-methyl-1,3-dipropylxanthine (WO 92/06976)

Melting point: 164.8-166.2° C. (Recrystallization from 2-propanol/water)

Elemental analysis: C₂₂H₂BN₄O₄

Calcd. (%): C, 64.06; H, 6.84; N, 13.58

Found (%): C 64.06, H 6.82, N 13.80

IR(KBr) νmax(cm⁻¹): 1692, 1657 NMR(DMSO-d₆, 270 MHz) δ(ppm): 7.60(1H, d, J=15.8 Hz), 7.40(1H, d, J=2.0 Hz), 7.28(1H, dd, J=2.0, 8.4 Hz), 7.18(1H, d, J=15.8 Hz), 6.99(1H, d, J=8.4 Hz), 4.02(3H, s), 3.99(2H, t), 3.90-3.80(2H, m), 3.85(3H, s), 3.80(3H, s), 1.85-1.50(4H, m), 1.00-0.85(6H, m)

Compound 3: (E)-1,3-diethyl-8-(3-methoxy-4,5-methylenedioxy styryl)-7-methylxanthine (JP 6-211856 A)

Melting point: 201.5-202.3° C.

Elemental analysis: C₂₀H₂₂N₄O₅

Calcd. (%): C, 60.29; H, 5.57; N, 14.06

Found (%): C, 60.18; H, 5.72; N, 13.98

IR(KBr) νmax(cm⁻¹): 1694, 1650, 1543, 1512, 1433

NMR(DMSO-d₆, 270 MHz) 8(ppm): 7.58(1H, d, J=15.8 Hz), 7.23(1H, d, J=15.8 Hz), 7.20(1H, d, J=1.0 Hz), 7.09(1H, d, J=1.0 Hz), 6.05(2H, s), 4.09-4.02(2H, m), 4.02(3H, s), 3.94-3.89(2H, m), 3.89(3H, s), 1.25(3H, t, J=7.2 Hz), 1.13(3H, t, J=6.9 Hz)

Compound 4: (E)-8-(3,4,5-trimethoxystyryl)caffeine (JP 47-26516 B)

IR(KBr) νmax(cm⁻¹): 1702, 1667, 1508, 1432

NMR(DMSO-d₆, 270 MHz) δ(ppm): 7.61(1H, d, J=16.0 Hz), 7.25(1H, d, J=16.0 Hz), 7.09(2H, s), 4.03(3H, s), 3.85(6H, s), 3.71(3H, s), 3.45(3H, s), 3.21(3H, s)

MS(EI) 386(M⁺)

Hereinafter, the pharmacological activity of compound (I) is shown by the following Test Examples.

### TEXT EXAMPLE 1

### Action of Increasing Feed Intake by 4-Week Oral Administration

1. Preparation of a Solution Containing a Test Compound

Compound 1 was suspended in a 0.5% aqueous methyl cellulose solution to a final concentration of 0.6 mg/mL for 6 mg/kg dose, 3 mg/mL for 30 mg/kg dose, 16 mg/mL for 160 mg/kg dose, or 80 mg/mL for 800 mg/kg dose, and the resulting solution was used.

2. Animals Used

Male and female Crj:CD (SD) rats (SPF) at the age of 5 weeks were purchased from Nippon Charles River Co., Ltd., and were then fed and conditioned for 9 days. During the term, their body weight change and conditions were observed, and animals determined as in healthy conditions were used at the test. At the start of the use, the body weights of the male animals were within a range of 187 to 209 g, while those of the female animals were within a range of 147 to 173 g. During both of the conditioning and feeding term and the test term, the animals were fed with a solid feed for mouse and rat (CRF-1 15 kGy, Oriental Yeast Industry, Co., Ltd.) and with water, ad libitum.

3. Composition of Test Groups

As to the number of the animals used, 15 each of male and female animals were used for each group. As a negative control group, a group administered a 0.5% aqueous methyl cellulose solution alone was prepared. Based on the body weight at the termination of the conditioning and feeding term, the animals were randomly assigned to individual groups, so that the body weights were uniformly distributed among the resulting individual groups.

4. Administration Method and Administration Term

Oral administration was selected as the administration route. The solvent or a test compound-containing solution was administered once daily in the morning for 4 weeks in a dose of 1 mL per 100 g of body weight.

5. Test Results

The feed intake of the test animals was measured on days 7, 14, 21 and 28. The measured values were tested for homoscedasticity by the Bartlett's test. When the variance was homogeneous, one-way layout analysis of variance was performed; in the case that significance was observed, herein, the control group and the individual dosed groups were tested by the Dunnett's test. When the variance was not homogeneous, the Kruskal-Wallis's rank test was performed; in the case that significance was observed among the groups, the Dunnett's test was conducted.

The results are shown in Table 2.

TABLE 2

| | Dose | | Feed intake (g ± SD) | | | |
|---|---|---|---|---|---|---|
| Compound | (mg/kg, po) | Sex | day 7 | day 14 | day 21 | day 28 |
| Solvent control | — | male | 23.50 ± 1.53 | 24.62 ± 1.96 | 25.39 ± 1.91 | 25.12 ± 2.09 |
| (0.5% methyl | | | | | | |
| cellulose- | | | | | | |
| dosed group) | | | | | | |
| 1 | 6 | male | 21.45 ± 1.28 | 23.97 ± 1.21 | 24.96 ± 1.58 | 25.23 + 1.80 |
| 1 | 30 | male | 23.54 ± 2.15 | 26.12 ± 2.03 | 27.03 ± 2.07 | 27.37 ± 2.56* |
| 1 | 160 | male | 23.94 ± 1.58 | 26.20 ± 1.77 | 27.31 ± 1.74* | 27.81 ± 2.47** |
| 1 | 800 | male | 22.84 ± 1.81 | 25.40 ± 2.42 | 26.67 ± 2.19 | 27.03 ± 2.37 |
| Solvent control | — | female | 16.66 ± 1.29 | 18.09 ± 1.54 | 18.47 ± 1.91 | 18.14 ± 1.56 |
| (0.5% methyl | | | | | | |
| cellulose- | | | | | | |
| dosed group) | | | | | | |
| 1 | 6 | female | 15.93 ± 1.09 | 18.03 ± 1.37 | 18.76 ± 1.18 | 18.80 ± 1.46 |
| 1 | 30 | female | 17.24 ± 1.19 | 19.38 ± 1.74 | 19.93 ± 2.08 | 20.50 ± 1.96** |
| 1 | 160 | female | 17.73 ± 1.40 | 19.56 ± 1.68* | 20.56 ± 1.73* | 20.87 ± 2.04** |
| 1 | 800 | female | 17.30 ± 0.99 | 19.19 ± 1.44 | 20.43 ± 1.69* | 21.13 ± 1.76** |

*P ≦ 0.05 (compared with the control group)
**P ≦ 0.01 (compared with the control group)

The test results show that the 4-week administration of Compound 1 increased the feed intake both in the males and in the females.

### TEST EXAMPLE 2

### Action of Increasing Feed Intake and Body Weight by 4-Week Oral Administration

1. Preparation of a Solution Containing a Test Compound

Compound 1 was suspended in a 0.5% aqueous methyl cellulose solution to a final concentration of 20 mg/mL for 200 mg/kg dose or 40 mg/mL for 400 mg/kg dose, and the resulting solution was used.

2. Animals Used

Male and female Crj:CD (SD) rats (SPF) at the age 5 of weeks were purchased from Nippon Charles River Co., Ltd., and were then fed and conditioned for 7 days. During the term, their body weight change and conditions were observed, and animals determined as in healthy conditions were used at the test. At the start of the use, the body weights of the male animals were within a range of 177.4 to 193.6 g, while those of the female animals were within a range of 141.1 to 160.3 g. During both of the conditioning and feeding term and the test term, the animals were fed with a solid feed for mouse and rat [FR-2, Funabashi Agricultural Farm Co., Ltd.] and with water ad libitum.

3. Composition of Test Groups

As to the number of the animals used, 5 each of male and female animals were used for each group. As a negative control group, a group administered a 0.5% aqueous methyl cellulose solution alone was prepared. Based on the body weight at the termination of the conditioning and feeding term, the animals were randomly assigned to individual groups, so that the body weights were uniformly distributed among the resulting individual groups.

4. Administration Method and Administration Term

Oral administration was selected as the administration route. The solvent or the test compound was administered once daily in the morning for 4 weeks in a dose of 1 mL per 100 g of body weight.

5. Test Results

The body weights and feed intake of the test animals were measured on days 0, 7, 14, 21 and 28. The individual measured values were tested, based on the same standards as in the Test Example 2.

The results are shown in Table 3 (body weight change) and Table 4 (feed intake change).

TABLE 3

| | Dose | | Body weight (g ± SD) | | | | |
|---|---|---|---|---|---|---|---|
| Compound | (mg/kg, po) | Sex | day 0 | day 7 | day 14 | day 21 | day 28 |
| Solvent control | — | male | 188.0 ± 4.0 | 240.9 ± 6.7 | 301.5 ± 16.2 | 353.2 ± 19.3 | 396.4 ± 23.8 |
| (0.5% methyl | | | | | | | |
| cellulose- | | | | | | | |
| dosed group) | | | | | | | |
| 1 | 200 | male | 191.2 ± 2.4 | 247.3 ± 4.8 | 313.5 ± 12.1 | 371.5 ± 23.9 | 419.2 ± 28.0 |
| 1 | 400 | male | 189.5 ± 4.4 | 242.5 ± 10.7 | 304.8 ± 15.7 | 361.9 ± 23.8 | 415.6 ± 29.2 |
| Solvent control | — | female | 149.7 ± 4.1 | 174.1 ± 4.6 | 199.5 ± 6.3 | 219.7 ± 8.1 | 237.2 ± 10.3 |
| (0.5% methyl | | | | | | | |
| cellulose- | | | | | | | |
| dosed group) | | | | | | | |
| 1 | 200 | female | 151.0 ± 5.7 | 178.6 ± 7.6 | 206.0 ± 7.1 | 232.5 ± 13.4 | 255.7 ± 17.3 |
| 1 | 400 | female | 152.9 ± 4.5 | 178.1 ± 3.3 | 207.8 ± 11.5 | 236.5 ± 16.3 | 259.3 ± 14.3 |

TABLE 4

| | Dose | | Feed intake (g ± SD) | | | | |
|---|---|---|---|---|---|---|---|
| Compound | (mg/kg, po) | Sex | day 0 | day 7 | day 14 | day 21 | day 28 |
| Solvent control | — | male | 20.3 ± 0.8 | 27.8 ± 1.5 | 28.3 ± 3.2 | 26.9 ± 2.2 | 29.2 ± 1.5 |
| (0.5% methyl | | | | | | | |
| cellulose- | | | | | | | |
| dosed group) | | | | | | | |
| 1 | 200 | male | 20.1 ± 1.0 | 28.4 ± 1.5 | 29.9 ± 1.6 | 30.2 ± 3.5 | 31.0 ± 1.1 |
| 1 | 400 | male | 20.7 ± 1.2 | 27.9 ± 2.5 | 30.2 ± 3.8 | 30.3 ± 3.9 | 34.8 ± 4.4 * |
| Solvent control | — | female | 13.5 ± 1.9 | 19.0 ± 1.0 | 19.0 ± 1.1 | 16.5 ± 1.9 | 17.5 ± 2.1 |
| (0.5% methyl | | | | | | | |
| cellulose- | | | | | | | |
| dosed group) | | | | | | | |
| 1 | 200 | female | 13.4 ± 1.7 | 20.2 ± 1.7 | 21.8 ± 1.7 | 20.9 ± 2.6 * | 23.8 ± 3.0 ** |
| 1 | 400 | female | 14.6 ± 1.7 | 19.5 ± 2.2 | 21.1 ± 2.9 | 22.0 ± 2.6 ** | 24.5 ± 2.5 ** |

* P ≦ 0.05(compared with the control group)
** P ≦ 0.01(compared with the control group)

The test results show that the 4-week administration of Compound 1 increased the feed intake and body weight both in ales and in the females.

### TEST EXAMPLE 3

### Acute Toxicity Test

Test compounds were orally administered to groups of dd-strain male mice weighing 20±1 g, each group consisting of three mice. Seven days after the administration, the mortality was observed to determine a minimum lethal dose (MLD) of each compound.

The MLD value of Compound 1 was greater than 1000 mg/kg.

Compound (I) or pharmaceutically acceptable salts thereof have an action of increasing feed intake and body weight. Thus, compound (I) or pharmaceutically acceptable salts are useful as a therapeutic agent for eating disorders, such as anorexia nervosa (cibophobia, absolute anorexia nervosa).

Compound (I) or pharmaceutically acceptable salts thereof can be used as such or in the form of various pharmaceutical compositions. The pharmaceutical compositions of the present invention can be prepared by uniformly mixing an effective amount of compound (I) or a pharmaceutically acceptable salt thereof as an active ingredient with pharmaceutically acceptable carriers. The pharmaceutical compositions are preferably in a unit dosage form suitable for rectal administration, oral or parenteral (including subcutaneous, intravenous and intramuscular administration) administration, etc.

For preparing a pharmaceutical composition for oral administration, any useful pharmaceutically acceptable carriers can be used. For example, liquid preparations for oral administration such as suspension and syrup can be prepared using water; sugars such as sucrose, sorbitol or fructose; glycols such as polyethylene glycol or propylene glycol; oils such as sesame oil, olive oil or soybean oil; preservatives such as a p-hydroxybenzoate; flavors such as strawberry flavor or peppermint, etc. Powder, pills, capsules and tablets can be prepared using excipients such as lactose, glucose, sucrose or mannitol; disintegrators such as starch or sodium alginate; lubricants such as magnesium stearate or talc; binders such as polyvinyl alcohol, hydroxypropyl cellulose or gelatin; surfactants such as fatty acid esters; plasticizers such as glycerin, etc. Tablets and capsules are the most useful oral unit dosage because of the readiness of administration. For preparing tablets and capsules, solid pharmaceutical carriers are used.

Injections can be prepared using carriers such as distilled water, a salt solution, a glucose solution or a mixture of a salt solution and a glucose solution. The preparation can be prepared in the form of a solution, suspension or dispersion by using a suitable auxiliary according to a conventional method.

Compound (I) or a pharmaceutically acceptable salt thereof can be administered orally in the pharmaceutical composition described above or parenterally as the injection or the like. The effective dose and administration schedule vary depending on the mode of administration, the age, the weight of a patient, symptoms of the disease, etc. However, generally, compound (I) or a pharmaceutically acceptable salt thereof is administered in a dose of 1 to 900 mg/60 kg/day, preferably, in a dose of 1 to 200 mg/60 kg/day, at one time or in several parts.

### BEST MODE FOR CARRYING OUT THE INVENTION

Certain embodiments of the present invention are described in the following examples.

### EXAMPLE 1

### Tablets

Tablets having the following composition are prepared in a conventional manner.

Compound 1 (40 g) is mixed with 286.8 g of lactose and 60 g of potato starch, followed by addition of 120 g of a 10% aqueous solution of hydroxypropyl cellulose. The resultant mixture is kneaded, granulated, and then dried by a conventional method. The granules are refined to give granules used to make tablets. After mixing the granules with 1.2 g of magnesium stearate, the mixture is formed into tablets each containing 20 mg of the active ingredient by using a tablet maker (Model RT-15, Kikusui) having pestles of 8 mm diameter.

Composition

| | | |
|---|---|---|
| Compound 1 | 20 | mg |
| Lactose | 143.4 | mg |
| Potato Starch | 30 | mg |
| Hydroxypropyl Cellulose | 6 | mg |
| Magnesium Stearate | 0.6 | mg |
| | 200 | mg |

### EXAMPLE 2

### Capsules

Capsules having the following composition are prepared in a conventional manner.

Compound 1 (200 g) is mixed with 995 g of Avicel and 5 g of magnesium stearate. The mixture is put in hard capsules No. 4 each having a capacity of 120 mg by using a capsule filler (Model LZ-64, Zanashi) to give capsules each containing 20 mg of the active ingredient.

Composition

| | | |
|---|---|---|
| Compound 1 | 20 | mg |
| Avicel | 99.5 | mg |
| Magnesium Stearate | 0.5 | mg |
| | 120 | mg |

### EXAMPLE 3

### Injections

Injections having the following composition are prepared in a conventional manner.

Compound 1 (1 g) is dissolved in 100 g of purified soybean oil, followed by addition of 12 g of purified egg yolk lecithin and 25 g of glycerin for injection. The resultant mixture is made up to 1,000 ml with distilled water for injection, thoroughly mixed, and emulsified by a conventional method. The resultant dispersion is subjected to aseptic filtration by using 0.2 μm disposable membrane filters, and then aseptically put into glass vials in 2 ml portions to give injections containing 2 mg of the active ingredient per vial.

Composition

| | | |
|---|---|---|
| Compound 1 | 2 | mg |
| Purified Soybean Oil | 200 | mg |
| Purified Egg Yolk Lecithin | 24 | mg |
| Glycerine for Injection | 50 | mg |
| Distilled Water for Injection | 1.72 | ml |
| | 2.00 | ml |

### EXAMPLE 4

### Anal Suppository

Formulations for rectal administration having the following composition are prepared in a conventional manner.

Witepsol® H15 (678.8 g, manufactured by Dynamit Nobel, Ltd.) and Witepsol® E75 (290.9 g, manufactured by Dynamit Nobel, Ltd.) are melted at 40 to 50° C. In the resulting molten mixture are uniformly mixed and dispersed Compound 1 (2.5 g), potassium dihydrogen phosphate (13.6 g) and disodium hydrogen phosphate (14.2 g). The resulting dispersion is poured into plastic suppository molds, and gradually cooled to give anal suppositories containing 2.5 mg of the active ingredient per formulation.

Composition

| | | |
|---|---|---|
| Compound 1 | 2.5 | mg |
| Witepzol H15 | 678.8 | mg |
| Witepzol E75 | 290.9 | mg |
| Potassium dihydrogen phosphate | 13.6 | mg |
| Disodium hydrogen phosphate | 14.2 | mg |
| | 1000 | mg |

### INDUSTRIAL APPLICABILITY

The present invention provides therapeutic agents for eating disorders, comprising a xanthine derivative or a pharmaceutically acceptable salt thereof as an active ingredient.

1. A method for therapeutically treating eating disorders, which comprises administering to a person in need of an increase in appetite an effective amount of a xanthine derivative to provide an increase of appetite, wherein the xanthine derivative is represented by formula (IA): 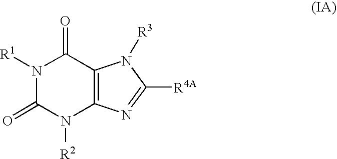
wherein R¹, R² and R³ independently represent hydrogen, lower alkyl, lower alkenyl or lower alkynyl; R^{4A} represents the following group: 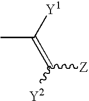
wherein Y¹ and Y² independently represent hydrogen, halogen or lower alkyl, and Z represents substituted or unsubstituted aryl, or the following group: 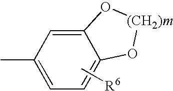
wherein m is an integer of 1 to 3 and R⁶ represents hydrogen, hydroxy, lower alkyl, lower alkoxy, halogen, nitro or amino, or a substituted or unsubstituted heterocyclic group, or a pharmaceutically acceptable salt thereof.

2. A method for therapeutically treating anorexia, which comprises administering to a person in need of an increase in appetite an effective amount of a xanthine derivative to provide an enhancement of appetite, wherein the xanthine derivative is represented by formula (I): 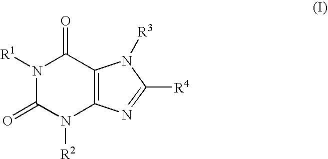
wherein R¹, R² and R³ independently represent hydrogen, lower alkyl, lower alkenyl or lower alkynyl; R⁴ represents cycloalkyl, —(CH₂)ₙ—R⁵, wherein R⁵ represents substituted or unsubstituted aryl, or a substituted or unsubstituted heterocyclic group, and n is an integer of 0 to 4, or the following group: 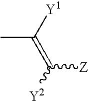
wherein Y¹ and Y² independently represent hydrogen, halogen or lower alkyl, and Z represents substituted or unsubstituted aryl, or the following group: 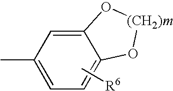
wherein m is an integer of 1 to 3 and R⁶ represents hydrogen, hydroxy, lower alkyl, lower alkoxy, halogen, nitro or amino, or a substituted or unsubstituted heterocyclic group, or a pharmaceutically acceptable salt thereof.

3. The method of claim 1, wherein R^{4A} is the following group: 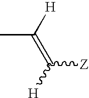
wherein Z has the same meaning as defined above, or a pharmaceutically acceptable salt thereof.

4. The method of claim 2, wherein R⁴ is the following group: 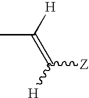
wherein Z has the same meaning as defined above, or a pharmaceutically acceptable salt thereof.

5. A method for enhancing appetite, which comprises administering to a person in need of an enhancement in appetite an effective amount of a xanthine derivative to provide an enhancement of appetite, wherein the xanthine derivative is represented by formula (I): 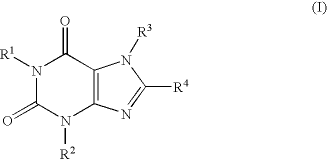
wherein R¹, R² and R³ independently represent hydrogen, lower alkyl, lower alkenyl or lower alkynyl; R⁴ represents cycloalkyl, —(CH₂)ₙ—R⁵, wherein R⁵ represents substituted or unsubstituted aryl, or a substituted or unsubstituted heterocyclic group, and n is an integer of 0 to 4, or the following group: 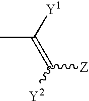
wherein Y¹ and Y² independently represent hydrogen, halogen or lower alkyl, and Z represents substituted or unsubstituted aryl, or the following group: 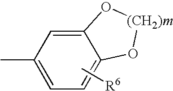
wherein m is an integer of 1 to 3 and R⁶ represents hydrogen, hydroxy, lower alkyl, lower alkoxy, halogen, nitro or amino, or a substituted or unsubstituted heterocyclic group, or a pharmaceutically acceptable salt thereof.

6. The method of claim 5, wherein R⁴ is the following group: 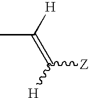
wherein Z has the same meaning as defined above, or a pharmaceutically acceptable salt thereof.

7. The method of claim 1, wherein said person has an eating disorder and is in need of the increase in appetite.

8. The method of claim 2, wherein said person has anorexia and is in need of the increase in appetite.
